(19) United States
(12) Patent Application Publication
(10) Pub. No.: US 2008/0262473 A1
(43) Pub. Date: Oct. 23, 2008

(54) Locating a Catheter Tip Using a Tracked Guide

(75) Inventors: Giora Kornblau, Binyamina (IL); Shlomi Ben-Ari, Binyamina (IL)

Correspondence Address: Martin D. Moynihan;PRTSI, P.O.Box 16446, Arlington, VA, 22215, US

(73) Assignee: Navotek Medical Ltd., Yokneam (IL)

(21) Appl. No.: 11/665,844

(22) PCT Filed: Oct. 19, 2005
(86) PCT No.: PCT/IL2005/001101

§ 371 Date: Apr. 19, 2007

Related U.S. Application Data
(60) Provisional application No. 60/619,792, filed on Oct. 19, 2004.
(60) Provisional application No. 60/619,897, filed on Oct. 19, 2004.
(60) Provisional application No. 60/619,898, filed on Oct. 19, 2004.

(30) Foreign Application Priority Data
Aug. 11, 2005 (IL) PCT/IL2005/000871

(51) Int. Cl. A61M 25/095

(52) U.S. Cl. 604/529

(57) ABSTRACT

A method of determining a position of a second object which travels along a first object. The method comprising determining a position for a first object; determining a linear displacement of a second object relative to the first object; and ascertaining a position of said second object based upon said relative linear displacement and said position of said first object.

### RELATED APPLICATIONS

The present application claims the benefit under 35 USC 119(e) of U.S. Provisional Application No. 60/619,898 filed on Oct. 19, 2004; entitled “Tracking a Catheter Tip by Measuring its Distance From a Tracked Guide Wire Tip”, U.S. Provisional Application No. 60/619,792 filed on Oct. 19, 2004, entitled “Using a Catheter or Guidewire Tracking System to Provide Positional Feedback for an Automated Catheter or Guidewire Navigation System”, U.S. Provisional Application No. 60/619,897 filed on Oct. 19, 2004 and entitled “Using a Radioactive Source as the Tracked Element of a Tracking System”, the disclosures of all of these application are incorporated herein by reference. This application is also a continuation in part of PCT/IL2005/000871 filed on Aug. 11, 2005, entitled “Localization of a Radioactive Source within a Body of a Subject” which claims the benefit under section 119(e) of U.S. Provisional Application No. 60/600,725, filed on Aug. 12, 2004, entitled “Medical Navigation System Based on Differential Sensor”, the disclosures of which are also incorporated herein by reference.

### FIELD OF THE INVENTION

The present invention relates to locating medical tools, for example, catheters inside the body.

### BACKGROUND OF THE INVENTION

There are numerous medical techniques which rely upon navigation of an operable tool deep within the body. In many cases, the tool is inserted from a distal site, (e.g. femoral blood vessel), and navigated a great distance through the body to a target location such as a coronary artery. These techniques rely upon tracking technologies to determine a position of the tool.

Some tracking technologies rely upon direct establishment of a 3D position of a tool within a body using a position sensor on the tool.

Additional tracking technologies rely upon establishing a map of a portion of a body (e.g. an arterial tree) and overlaying an image/position of a tool on the map. For example, Lengyel et al. (http://www.graphics.cornell.edu/pubs/1995/LGP95.pdf.), the disclosure of which is incorporated herein by reference, teaches linear measurement of catheter displacement and comparison to arterial tomography data.

### SUMMARY OF THE INVENTION

An aspect of some embodiments of the present invention relates to determining a position of a second object which travels along a first object, or a path traveled by it, using a determined position of the first object and a relative linear displacement between the objects. Optionally, a series of positions define a path of the first object. In an exemplary embodiment of the invention, the second object is a catheter and the first object is a catheter guidewire. Optionally, the guidewire carries a tracking source at or near its tip. Optionally, the tracking source is a radioactive source. Optionally the position is a 3D or a 2D position.

An aspect of some embodiments of the present invention relates to an intrabody medical system which employs machine readable markings to determine relative linear displacement of a first object and a second object. Optionally, the machine readable markings employ a binary code, for example a bar code. In an exemplary embodiment of the invention, a second object travels along a first object. In an exemplary embodiment of the invention, the system includes two objects, each marked with machine readable markings. In an exemplary embodiment of the invention, a sensor on one object and a reader on the other object are employed. In an exemplary embodiment of the invention, a single sensor is attached to both objects.

According to some aspects of the invention, there is provided, a method of determining a position of a second object which travels along a first object. The method comprising:

(a) determining a position for a first object;
(b) determining a linear displacement of a second object relative to the first object; and
(c) ascertaining a position of said second object based upon said relative linear displacement and said position of said first object.

Optionally, the method additionally includes measuring a linear displacement of said first object.

Optionally, said linear displacement of said first object and said second object linear displacement are each determined relative to a defined point.

Optionally, the defined point employed for said first object and said second object are a single defined point.

Optionally, the defined point employed for said first object and said second object are two separate defined points and the method additionally includes computing a linear distance between a first defined point employed in determining said first object linear displacement and a second defined point employed in determining said second object linear displacement and correcting said second object linear displacement value by said linear distance.

Optionally, said determining said linear displacement and said position for the first object is repeated to determine a series of linear displacements with correlated positions, said series of correlated positions defining a path.

Optionally, said determining a linear displacement value for said second object is relative to a defined point on said first object.

Optionally, said second object is a catheter.

Optionally, said first object is a guidewire.

Optionally, said guidewire carries a tracking source at or near its distal tip.

Optionally, said tracking source is a radioactive source.

According to some aspects of the invention, there is provided, a system for performing a medical procedure. The system comprising at least two axially extensible members at least partially insertable within a body, at least one of said axially extensible members marked with an array of machine readable markings configured to aid in determination of relative linear displacement along a common path of said at least two axially extensible members with respect to one another.

Optionally, said machine readable markings designate length increments.

Optionally, the system additionally includes a tracking source positioned at a distal portion of one of said axially extensible members.

Optionally, said tracking source is a radioactive source.

Optionally, said machine readable markings include a binary code.

Optionally, said axially extensible members include a catheter.

Optionally, said axially extensible members include a catheter guidewire.

Optionally, said catheter guidewire bears said array of machine readable markings.

According to some aspects of the invention, there is provided, a system for determining a position of a second object and a first object which travel along a common path. The system comprising:

(a) a first object comprising a tracking source, said first object subject to displacement along a path;
(b) a second object subject to displacement along said first object; and
(c) a displacement sensor designed and configured to determine a relative displacement of said second object and said first object along said path.

Optionally, the system additionally includes circuitry to convert said relative displacement of said second object and said first object along said path to a position of said second object.

Optionally, said tracking signal includes radioactive disintegrations.

Optionally, wherein said displacement sensor includes an optical sensing mechanism.

Optionally, wherein said optical sensing mechanism reads a binary code.

Optionally, wherein said displacement sensor includes a mechanical sensing mechanism.

Optionally, said first object includes a guidewire.

Optionally, said second object includes a catheter.

According to some aspects of the invention, there is provided, a method of determining a position of a second object which travels along a first object. The method comprising:

(a) determining a path traveled by at least one point on a first object;
(b) causing a second object to travel along said path while additionally determining a linear displacement of at least one point on said second object; and
(c) determining a position of a selected portion of the second object by calculating a progress along the path of the at least one point on first object.

Optionally, said calculation relies upon said linear displacement.

Optionally, the progress along the path of the first object is determined by:

(a) employing a fixed and known length for at least a portion of each of the first and second objects; and
(b) measuring a relative displacement of said at least one point on said second object along said first object.

Optionally, said measuring said relative displacement is conducted outside a body of a subject.

Optionally, the said linear displacement is determined by:

(a) defining a first object reference point at a known distance from a distal extremity of said first object;
(b) defining a second object reference point at a known distance from a distal extremity of said second object; and
(c) measuring a distance between said first object reference point and said second object reference point as a means of computing a relative position of said distal extremity of said first object and said distal extremity of said second object along said path.

Optionally, said first object reference point and said second object reference point are initially aligned in a same position.

Optionally, said second object is a catheter.

Optionally, said first object is a guidewire.

Optionally, said guidewire carries a tracking source at or near its distal tip.

Optionally, said tracking source is a radioactive source.

According to some aspects of the invention, there is provided, a guidewire comprising a source of radiation integrally formed with or attached to a distal portion thereof.

Optionally, said radiation is in the range of 0.01 mCi to 0.5 mCi, optionally 0.1 mCi or less.

Optionally, said detectable amount is 0.05 mCi or less.

Optionally, said isotope is Iridium-192.

### BRIEF DESCRIPTION OF FIGURES

In the Figures, identical structures, elements or parts that appear in more than one Figure are generally labeled with the same numeral in all the Figures in which they appear. Dimensions of components and features shown in the Figures are chosen for convenience and clarity of presentation and are not necessarily shown to scale. The Figures are listed below.

FIG. 1 is a schematic representation of operational components of a system according to an exemplary embodiment of the invention;

FIG. 2 is a diagram illustrating relative linear displacement measurement according to an exemplary embodiment of the invention;

FIG. 3 illustrates an exemplary optical mechanism for measuring relative linear displacement according to an exemplary embodiment of the invention;

FIG. 4 illustrates machine readable markings on a guidewire according to an exemplary embodiment of the invention; and

FIG. 5 illustrates an exemplary mechanical mechanism for measuring relative linear displacement according to an exemplary embodiment of the invention.

### DETAILED DESCRIPTION OF EMBODIMENTS

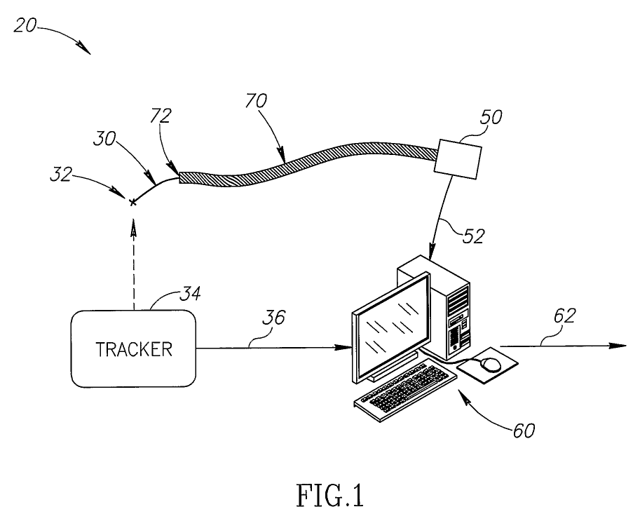

FIG. 1 illustrates a system 20 for determining a position of a second object which travels along a first object using a determined position of the first object and a relative linear displacement, in accordance with an exemplary embodiment of the invention. Optionally, a series of positions define a path of the first object. In the pictured exemplary embodiment of the invention, the second object is a catheter 70 and the first object is a guidewire 30. According to various embodiments of the invention, the position may be a 3D or a 2D position.

### Measuring Position of the First Object:

In an exemplary embodiment of the invention guidewire 30, serving as the first object, carries a tracking source. The tracking source may be any object for which a position sensing system can determine a position. According to various embodiments of the invention, the tracking source may either provide or monitor a signal. Optionally, the tracking source is located at any location on guidewire 30. In an exemplary embodiment of the invention, the tracking source is located at or near a distal tip 32 of guidewire 30. In exemplary embodiments of the invention, the tracking source includes one or more of a radioactive source, an RF transmitter and/or receiver, and/or a magnet.

Determination of a position of origin of an RF signal relative to one or more receivers is generally known in the art and one of ordinary skill in the art will be able to incorporate any known method into the context of the present invention. Some methods are based upon knowledge of signal strength at point of origin. These methods may be employed to determine a position of an RF receiver and/or RF signal source located on guidewire 30. Similar methods may be employed to track a magnet.

Examples of radioactive sources and detection systems for tracking them are described in co-pending application PCT/IL2005/000871, the disclosure of which is fully incorporated herein by reference. The described tracking systems rely on sensors to determine an angle between a known sensor position and the tracked source. Each of the sensors employs one or more walls to cause differential distribution of emissions from a radioactive source on a sensing area, the distribution varying with angle.

One example of a sensor suited for use in the context of the present invention employs two sensors separated by a perpendicular wall so that when the wall is pointed at the source, both sensors receive the same amount of incident radiation. When the detector and wall are rotated, the wall preferentially shadows one of the sensors and causes unequal distribution of the incident radiation. This configuration relies upon maximum detection of incident radiation and equal distribution of that radiation among the two sensors to indicate a correct angular direction towards the source. Additional configurations with two or more walls are also disclosed in application PCT/IL2005/000871.

Calculation of an intersection between two, optionally three, optionally four or more directions provides a location for the tracked source. In an exemplary embodiment of the invention, a tracking unit 34 periodically, optionally continuously, ascertains and records a position of the tracking source at or near distal tip 32 of guidewire 30. Alternatively or additionally, position of distal tip 32 may be ascertained using an imaging device.

The ascertained position of distal tip 32 of guidewire 30 is optionally expressed as a path of 3D position co-ordinates. Sampling density of the tracking unit 34 is related to accuracy of the determined path. Sampling density of tracking unit 34 may optionally be expressed in measurements per unit time and/or measurement per unit distance traveled by the tracked source. In an exemplary embodiment of the invention, tracking unit 34 computes a location of tip 32 of guidewire 30 once per second, optionally 10 times per second, optionally 20 times per second, optionally 50 times per second or more. Typically, as sampling density increases, the need for interpolation decreases. In an exemplary embodiment of the invention, the ascertained position may be incorporated into a position output signal 36, for example a signal 36 including a time stamp. The time stamp indicates at what time the position was detected. The time may be relative time measured from an arbitrary zero time point or clock time. Optionally, output signal 36 may be expressed as, for example time+2D position (t, X, Y) or time+3D position (t, X, Y, Z). The time stamp permits correlation with other independently acquired data as detailed hereinbelow.

Output signal 36 is optionally communicated to a computer 60. In an exemplary embodiment of the invention, output signal 36 may be converted by computer 60 to a plot of 3D position as a function of time. The term computer, as used in this specification and the accompanying claims, includes computational circuitry, including but not limited to an ASIC.

Although use of a radioactive tracking source has been used as an example, embodiments which rely upon other positioning systems, such as those employing one or more of radioactive disintegrations, radiofrequency energy, ultrasound energy, electromagnetic energy, NMR, CT, fluorography may be used, and are within the scope of the invention. Optionally, static and/or quasi-static electromagnetic fields are employed for tracking.

### Measuring Linear Displacement of the First Object:

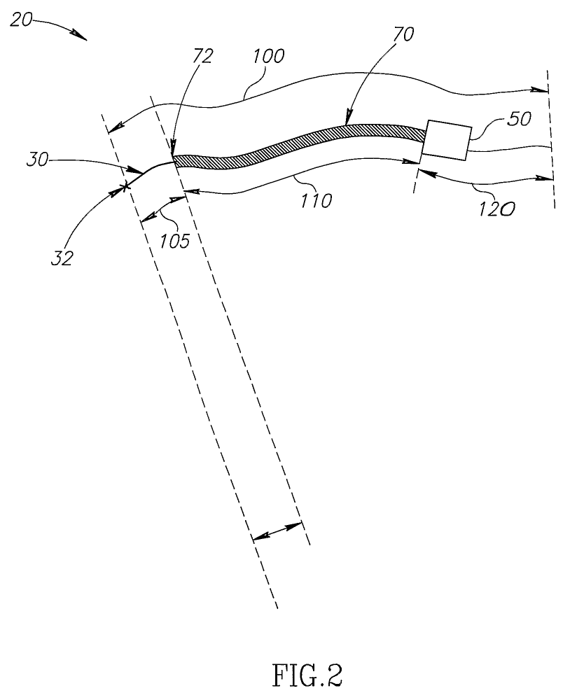

Referring now to FIG. 2, in an exemplary embodiment of the invention, a linear displacement sensor 50 determines a linear displacement 100 of tip 32 of guidewire 30 and expresses it as a linear displacement value. Displacement sensor 50 may operate according to various mechanisms according to alternate exemplary embodiments of the invention as detailed hereinbelow. In an exemplary embodiment of the invention, measured displacement of the first object is aligned to its measured position so that position may be expressed as a function of linear displacement.

In an exemplary embodiment of the invention, sensor 50 does not serve as a drive mechanism for guidewire 30; rather, sensor 50 registers the passage of guidewire 30 as the guidewire passes therethrough. In an exemplary embodiment of the invention, sensor 50 is incorporated into a drive mechanism for guidewire 30. Optionally, sensor 50 is fixed at a known location, for example by being positioned near a port serving as an entry point to a femoral artery being used in a catheterization procedure. Fixation at a known location may be accomplished, for example, by strapping a housing of sensor 50 to a leg of a patient or by attaching the housing of sensor 50 to an operating table. In an exemplary embodiment of the invention, the position of sensor 50 is arbitrarily defined as zero displacement with regard to guidewire 30.

In some embodiments of the invention, displacement sensor 50 relies upon a first sensing mechanism (e.g. optical sensing) to measure linear displacement of guidewire 30 and a second sensing mechanism (e.g. mechanical sensing) to measure linear displacement of catheter 70.

In an exemplary embodiment of the invention, displacement sensor 50 relies upon a similar mechanism to measure displacement of both catheter 70 and guidewire 30. In an exemplary embodiment of the invention, displacement of catheter 70 is measured relative to guidewire 30, optionally using a machine readable code on guidewire 30. Optionally, analytic circuitry 60, which may be, for example, a computer, computes distance 105 between guidewire tip 32 and catheter tip 72 is located in sensor 50. Alternatively or additionally, sensor 50 may be fixed to guidewire 30 and/or catheter 70.

In an exemplary embodiment of the invention, calibration is accomplished by reading position on a common scale, such as code 38 of guidewire 30. According to this embodiment a guidewire 30 and catheter 70 each having a known length are initially aligned so that an initial distance 105 between guidewire tip 32 and catheter tip 72 is known. For example, a guidewire 30 with a machine readable code marked in mm on a 1000 mm section of guidewire might be employed. Sensor 50 is attached to a proximal end of catheter 70 and held in a fixed position arbitrarily defined as 0 displacement. Guidewire 30 is moved 100 mm into the body and sensor 50 reads this displacement using the machine readable code on guidewire 30. Distance 10 has been increased by 100 mm at this stage. When displacement of catheter 70 begins, sensor 50 is displaced along guidewire 30. As this occurs, sensor 50 counts down using the machine readable code. For example, if catheter 70 advances 25 mm, sensor 50 would register a 75 position mm according to the code. Distance 105 is reduced by 25 mm, so that it is only 75 mm greater than its initial value. Alternatively or additionally, sensor 50 records its own motion relative to 0 displacement.

In an exemplary embodiment of the invention, an operator of system 20 may advance catheter 70 rapidly along guidewire 30. Optionally, an output 62 reflecting distance 105 between catheter tip 72 and guidewire tip 32 alerts the operator when to slow down so that the target may be approached slowly. Output 62 may be displayed visually or through an audio device (e.g. simulated speech) as a distance. Alternatively or additionally, a warning indicator may alert an operator when distance 105 drops below a preset limit. The warning indicator may be visible (e.g. an indicator light or icon on a display screen) and/or audible (e.g. bell, buzzer or simulated speech).

In an exemplary embodiment of the invention, two or more targets, such as arterial plaques, are designated along a single path as guidewire 30 advances. In an exemplary embodiment of the invention, target designation is based upon alignment with image data. Optionally, the image data is acquired concurrently with advancement of guidewire 30. Alternatively or additionally, previously acquired image data may be employed for target designation. Image data may be, for example fluoroscopy image data. Alternatively or additionally, targets may be sensed by increased resistance to advancement of tip 32 of guidewire 30. Optionally, an operator of the system may indicate target designations to computer 60. In an exemplary embodiment of the invention, targets are visible on a display screen of computer 60.

Tip 72 of catheter 70 may subsequently be brought into proximity to these targets based upon their relative displacements on a path defined by guidewire 30. Alternatively or additionally, a beginning and an end of a single plaque may be defined as separate targets in order to facilitate measurement of plaque length.

In an exemplary embodiment of the invention, targets are mapped, using displacement sensor 50 so that each target is expressed as one or more linear displacement values. Alternatively or additionally, targets are mapped using tracking unit 34 so that each target is expressed as one or more positions.

Displacement 100 may be supplied to computer 60 as a displacement output signal 52, optionally time stamped as detailed hereinabove. Optionally, computer 60 may calculate linear displacement of tip 32 of guidewire 30 as a function of time. Registration onto image data, such as fluoroscopy data and/or intravascular ultrasound data (IVUS) and/or arterial tomography data may be performed, for example as detailed hereinbelow.

Target designation, whether actively performed by an operator of the system, or passively implemented by alignment with image data containing visible targets, is useful to an operator of the system once the second object, such as a catheter, is deployed. Target designation may, for example, aid an operator in choosing speed for catheter advancement.

In some embodiments of the invention, tracking of multiple elements on a single path is facilitated. This tracking may optionally be concurrent or sequential. Multiple elements may be, for example, multiple radioactive sources and/or multiple radio-opaque markers as detailed hereinbelow.

In an exemplary embodiment of the invention, multiple sources may be tracked by multiple sensors. For example a radioactive source may be tracked by directionally sensitive radioactive sensors as detailed hereinabove and an RF source may be tracked by an RF sensing system. This may be useful, for example, in determining orientation of two points on a single object and/or coordinating activity of two separate objects. Alternatively or additionally, multiple radioactive sensors with different types of emissions may be concurrently tracked using sensors specific to each emission type. In an exemplary embodiment of the invention, only a single radioactive source is employed.

Optionally, linear displacement data may be calculated from a series of positions of the first object, for example tip 32 of guidewire 30. Accuracy of this calculated displacement may be increased by increasing the number of positions determined per unit displacement of the guidewire.

Alternatively or additionally, displacement of guidewire 30 and/or catheter 70 is not measured directly. In an exemplary embodiment of the invention, displacement of catheter 70 is measured relative to guidewire 30.

### Registration of Position and Linear Displacement of the First Object:

In an exemplary embodiment of the invention, position of tip 32 of guidewire 30 is expressed as a function of displacement. Optionally, computer 60 correlates position output signal 36 and displacement output signal 52. Optionally, expression of position as a function of displacement is useful in determining a position of a second object traveling along the same path for which only displacement data is available.

Optionally, output signals 36 and 52 are registered one with respect to the other so that a single displacement measurement indicates a single position. This facilitates a representation of the 3D position of tip 32 of guidewire 30 as a function of linear displacement.

Optionally, outputs 52 and 36 (linear displacement and position respectively) are registered one with respect to the other via correlation through an additional parameter, for example time. In some cases, time stamps on output signals 52 and 36 are not completely coincident and an interpolation algorithm is implemented to achieve registration. Interpolation may introduce inaccuracy into the registration.

In an exemplary embodiment of the invention, displacement output 52 has a greater sampling density than position output 36. In this case, displacements which more closely correspond to determined positions are more accurate. It is possible to determine an estimate of error for a position corresponding to any linear displacement value. Optionally, this estimate of error is displayed on a display of computer 60.

In an exemplary embodiment of the invention, the 3D position of tip 32 of guidewire 30 as a function of linear displacement is overlaid and/or registered upon an image or map of a body portion, for example an image or map of the brain. Alignment of the 3D position of tip 32 of guidewire 30 as a function of linear displacement with an image or map may be accomplished using any method known in the art. In an exemplary embodiment of the invention, this process produces an image, for example an image of the brain with a line representing the path of tip 32 of guidewire 30 overlaid on the image. Optionally, linear displacement data is displayed along the line, for example by use of hash marks and/or indicator numerals. This permits an operator to easily ascertain from a display screen the distance at which anatomical features of interest reside. Because the brain is static, image data acquired prior to the procedure and/or concurrent with the procedure may be employed.

Registration of the 3D position of tip 32 of guidewire 30 as a function of linear displacement on a dynamic organ, such as the heart, is more complicated. In order to facilitate accurate registration of the 3D position of tip 32 of guidewire 30 with image data of a dynamic body portion (e.g. a beating heart), additional registration may be employed. In an exemplary embodiment of the invention, a time stamped image output (e.g. fluorography or IVUS) is concurrently supplied to computer 60. Optionally, 3D position of tip 32 of guidewire 30, linear displacement of tip 32 of guidewire 30 and image data are all correlated one to another through time stamps. The result of this multiple correlation is a plot of 3D position of guidewire tip 32 as a function of linear displacement overlaid on a static map of a body portion. The static map represents a collection of relevant anatomical features from dynamic image data depicted relative to guidewire 30. This static map represents a path through the body which a second object, e.g. a catheter, may follow. Guidewire 30 serves as a track along the path.

Alternatively or additionally, once tip 32 of guidewire 30 has reached a desired target, the linear representation of 3D position as a function of time (even without regard to the static map) indicates a path which the second object will follow,

### Measuring Linear Displacement of the Second Object:

In an exemplary embodiment of the invention, linear displacement of a second object, for example a catheter 70 is measured. Although linear displacement of a single second object along a first object is described, two or more second objects may be made to travel along a first object according to some exemplary embodiments of the invention. As illustrated in FIGS. 1 and 2, a catheter 70 with a distal tip 72 may be made to travel along guidewire 30 so that catheter tip 72 approaches guidewire tip 32. Linear displacement of catheter 70 may be measured, for example, by a linear displacement sensor 50 as detailed hereinbelow. Sensor 50 may engage and/or propel catheter 70 by means of, for example, a mechanical mechanism such as a groove or mated sets of arcuate teeth. Optionally, catheter 70 and guidewire 30 may be measured by a single displacement sensor 50 or by different displacement sensors 50. For example, displacement of guidewire 30 may be sensed by an optical sensor 50 and displacement of catheter 70 along guidewire 30 may be sensed by a mechanical sensor 50 or the opposite.

In an exemplary embodiment of the invention, catheter 70 carries a sensor 50 which reads code 38 on guidewire 30. For example, a catheter 70 with a known length of 1000 mm might be positioned so that its proximal end is aligned with a proximal end of a guidewire 30 with a known length of 2000 mm. Using this example, tip 32 of guidewire 30 is advanced 767.3 mm into the body. This means that tip 72 of catheter is 232.7 mm from the entrance to the femoral artery. A position sensor 50 at the proximal end of catheter 70 reads code 38 and/or mechanically measures distance as catheter 70 advances along guidewire 30. This permits computation of distance 105 by subtraction.

### Ascertaining Position of the Second Object:

According to exemplary embodiments of the present invention, once a linear displacement of catheter tip 72 is known, its position (optionally 3D position) may be ascertained from the plot of position of first object (e.g. tip 32 of guidewire 30) as a function of linear displacement described above. This is possible because displacement of guidewire 30 and catheter 70 are along a common path. In some cases, for example cardiac catheterization, the entire path may move (e.g. during systolic contractions). However, this does not influence the relative linear displacement of the first and second objects.

Alternatively or additionally, it is possible to ascertain a distance 105 between catheter tip 72 and guidewire tip 32. Optionally, this distance may be employed to compute position co-ordinates of catheter tip 72, for example using the plot of position as a function of linear displacement of guidewire 30 described above.

In an exemplary embodiment of the invention, displacement of catheter tip 72 is measured relative to an arbitrary start point outside the body. This start point corresponds to a known position on guidewire 30 measured relative to guidewire tip 32. If the length of catheter 70 is known, it is possible to calculate distance 105. For example, if the known start point is 950 mm from guidewire tip 32, and a proximal end of catheter 70 advances 150 mm along guidewire 30, the proximal end of the catheter will be 800 mm from guidewire tip 32. If the catheter has a length of 600 mm, tip 72 of the catheter will have a distance 105 of 200 mm from tip 32 of the guidewire.

In an exemplary embodiment of the invention, displacement of catheter tip 72 is measured relative to guidewire tip 32. For example if a catheter 30 bearing a machine readable code is employed, a sensor 50 mounted at tip 72 of catheter 70 may read its distance 105 from tip 32 of catheter 30 directly.

In an exemplary embodiment of the invention, guidewire 30 may be subject to additional linear displacement 120 after catheter 70 is inserted in the body. Typically this additional linear displacement would alter incremental distance 105, but not cause a change in a calculated position of tip 72 of catheter 70. For example, if guidewire 30 has been inserted 100 cm as measured by sensor 50 and catheter 70 has been inserted 85 cm along the guidewire, a relative displacement 105 of 15 cm is calculated. The 3D position of tip 72 of catheter 70 can be determined from the plot of position as a function of linear displacement of guidewire 30 described above. If guidewire 30 is advanced an additional 10 cm, relative displacement 105 between tip 32 of guidewire 30 and tip 72 of catheter 70 will increase to 25 cm but the position of tip 72 of catheter 70 may optionally remain unchanged.

If guidewire 30 and catheter 70 are each advanced an additional 10 cm, relative displacement 105 between tip 32 of guidewire 30 and tip 72 of catheter 70 will remain unchanged but the position of tip 72 of catheter 70 may optionally advance along the path determined by guidewire tip 32.

In an exemplary embodiment of the invention, guidewire 30 advances first and catheter 70 follows along the guidewire. This results in a temporary increase in distance 105 which is later offset by advancement of catheter tip 72.

Alternatively or additionally, catheter tip 72 may advance, causing a decrease in distance 105 which is subsequently offset by an additional advance of guidewire tip 32.

In an exemplary embodiment of the invention, guidewire 30 and/or catheter 70 may be partially withdrawn and then advanced along a different path. This may be useful, for example, in a combined PTCA/angiography procedure or a procedure in which PCTA in multiple coronary arteries is performed.

Regardless of the order and/or amplitude of changes in displacement of catheter tip 72 and guidewire tip 32, relative displacement 105 and 3D position of tip 72 may be ascertained from position and displacement data of guidewire tip 32 or any other tracked part of the guidewire.

In an exemplary embodiment of the invention, the accuracy of a 3D position determined by tracking monitor 34 for guidewire tip 32 is high (e.g. within 2 mm, optionally 1 mm, optionally 0.5 mm, optionally 0.1 mm or less). In an exemplary embodiment of the invention, displacement measurements of catheter tip 72 and/or guidewire tip 32 do not significantly detract from this accuracy. As detailed hereinbelow, optical displacement sensing mechanisms with accuracy in the range of tens of nanometers have been described and mechanical sensing mechanisms with a sensitivity of 0.5 to 0.6 mm are well known in the art. Optionally, the linear displacement of the first object and the second object (e.g. guidewire 30 and catheter 70) are each accurate to within 2 mm, optionally 1 mm, optionally 0.5 mm, optionally 0.1 mm or less. In an exemplary embodiment of the invention, the total inaccuracy of distance 105 between catheter tip 72 and guidewire tip 32 does not exceed 2 mm, optionally 1.5 mm, optionally 1.0 mm, optionally 0.5 mm or less.

In some exemplary embodiments of the invention, the position of tip 32 of guidewire 30 is defined as a 2D position (i.e. X, Y). Alternatively or additionally, the position of tip 32 of guidewire 30 is defined as a 3D position (i.e. X, Y, Z). Optionally, the position plot of the first object is a 2D position plot or a 3D position plot.

In some exemplary embodiments of the invention, positions may be defined vectorially as a combination of angles and distances. For example, a position of tip 32 of guidewire 30 might be defined as a rotation angle, an elevation angle and a distance relative to a defined point. Optionally, the defined point may be an anatomical marker. For example, in an intracranial procedure, the incision point in the skull might be employed as a reference point and positions relative to this marker might be determined using near field RF transceivers.

Use of 2D positions may be useful, for example, in catheterization within a limb. Use of 3D positions may be useful, for example, in brain catheterization procedures, including but not limited to AVM treatment and/or intra-arterial stroke treatment as well as in cardiac catheterization procedures including, but not limited to angiography and/or angioplasty.

Optionally, output is displayed to a user, for example on a display screen, so that the relative positions of catheter tip 72 and guidewire tip 32 are visually comprehensible.

In an exemplary embodiment of the invention, a standard catheter 70 without a tracking source 70 is employed while accurate determination of a location of tip 72 thereof is achieved. Sensor 50 measuring displacement of catheter 70 permits a tracking source to be placed on guidewire 30

In an exemplary embodiment of the invention, a standard guidewire 30 with no machine readable code is employed and a mechanical displacement sensor 50 is employed to measure linear displacement of the guidewire.

### Exemplary Linear Displacement Sensor Types—Optical Sensor:

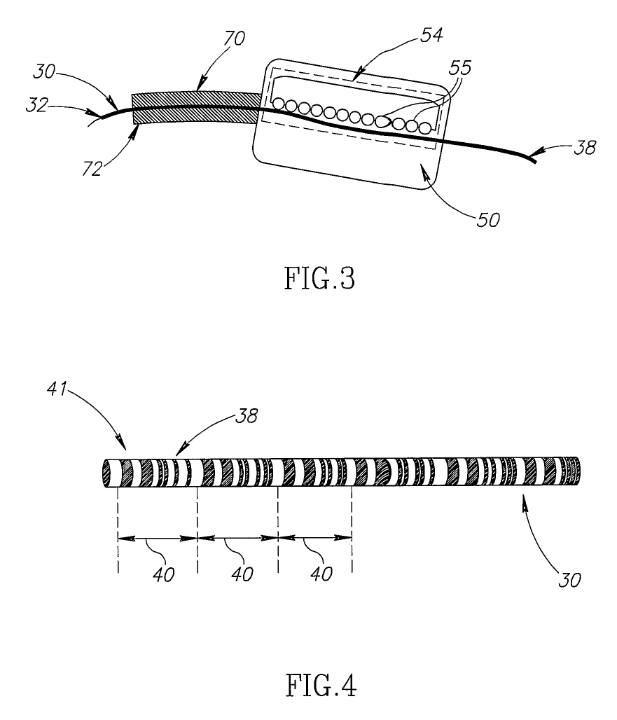

Referring now to FIG. 3, in an exemplary embodiment of the invention, linear displacement sensor 50 employs optical sensing means 54 such as, for example, one or more CCD elements 55 (e.g. CCD elements available from Hamamatsu Photonics K.K., Japan) to read a machine readable code 38 optically encoded on guidewire 30 and/or catheter 70. These markings may indicate, for example, how much of an object (e.g. guidewire 30 and/or catheter 70) has passed a given point. Mechanisms for optical sensing are well known to those of ordinary skill in the art and can be incorporated into the context of the present invention. Codes 38 may be relative codes which rely upon counting or absolute codes which permit determination of a displacement from a single reading. In an exemplary embodiment of the invention, a combination of absolute and relative codes is employed.

Referring now to FIG. 4, in an exemplary embodiment of the invention, code 38 includes segment indicators 40 of known length, optionally organized in groups of sub-segments. Optionally, sensor 50 has a reading frame which is longer than a sub-segment so that each sub-segment may be accurately read. Optionally, code 38 includes a start marking 40 which indicates an initial distance from tip 32 of guidewire 30. For example, the start code might be placed 1000 mm from tip 32 and code 38 might extend 500 mm along guidewire 30 away from tip 32 towards the proximal end. This permits a first increment of guidewire insertion to be accurately registered without incremental measurement. In some uses, the initial approach of the guidewire to the area of interest is not the subject of location analysis. For example, the exact position of tip 32 of guidewire 30 as it moves through the femoral artery towards the heart is of relatively little interest while the exact position of tip 32 of guidewire 30 once it is in the pulmonary artery system is of greater interest. Optionally, sub-segments (not shown) are also indicated. In an exemplary embodiment of the invention, code 38 is a bar code. In an exemplary embodiment of the invention, code 38 employs a unique pattern as a start code for each of segments 40.

In some exemplary embodiments of the invention, segments and/or sub-segments are sequentially counted. In some exemplary embodiments of the invention, each of segments 40 additionally includes a binary encoding of the segment number. For example, segment number 13 may be encoded in binary as 1101 which translates into a Black/White code of Black, Black, White, Black (assuming Black is 1). A code using a band width of 0.1 mm read by 100 CCD elements 55 each 0.1 mm wide can produce measurement accuracy of 0.1 or better. Optionally, light is supplied from an internal source such as an LED light source is sensor 50 illuminating code 38. Optionally, an LSB (least significant bit) of the segment code begins at a known distance from the start code, so that each digit line is registered to a correct position. In an exemplary embodiment of the invention, a line width of the start marking differs from the code pattern (e.g. each line may be 1.5 times wider) so that sensor 50 will not confuse a start marking with binary code. In an exemplary embodiment of the invention, line width corresponds to the width of CCD elements 55, except for start markings which are wider than CCD elements 55. Optionally, a start marking is defined as a mark which simultaneously registers in two CCD elements 55. Optionally, an error checking technique (e.g. parity mechanism) is used to reduce mistakes in interpreting the code. In an exemplary embodiment of the invention, code 38 may be read using a Vernier scale to increase accuracy. Optionally, positioning of CCD elements 55 may create the Vernier scale.

Alternatively or additionally, code 38 may employ Moire modulation of overlapping gratings to generate fringes. This technique can theoretically produce resolution in the range of 14 nm for a grating with 10 micron spacing. (Suezou Nakadate et al (2004) Meas. Sci. Technol. 1: 1462-1455). This article is fully incorporated herein by reference. In an exemplary embodiment of the invention, one set of gratings may be placed on or attached to a portion of guide wire 30. Alternatively or additionally, one set of gratings may be placed on or attached to a portion of catheter 70. Alternatively or additionally, one set of gratings may be interposed between CCD elements 55 and a portion of guidewire 30 and/or catheter 70.

In an exemplary embodiment of the invention, in order to estimate the absolute location of sensor 50 along guidewire 30, a position measurement algorithm installed on computer 60 (optionally an ASIC device) identifies the start pattern, and optionally measures its location within the sensor's image. The segment number is optionally deduced from the binary encoding. The location may then be calculated by multiplying the segment number by the segment length and adding the start pattern location within the image. Optionally, an array of many CCD elements 55 are employed so that the position of a border between two sequential segments within the distance covered by the array can be ascertained as it moves past the array. In an exemplary embodiment of the invention, sensor 50 operates on a straight portion of guidewire 30 and/or catheter 70 located outside of the body of a patient. This reduces measurement errors which might be caused by bending.

For example, if a segment's length is 10 mm, the algorithm may detect that the start pattern lays 7.3 mm from the image start (serves as the reference point), and the binary code indicates that this is segment number 46, then the absolute location of sensor 50 along the guidewire is (46)×10+7.3=467.3 mm. Optionally, the start signal is positioned a known distance from tip 32 of guidewire 30 as detailed hereinabove. In that case, the known distance must be added to the calculated displacement. Using the above example, a guidewire 30 with a start signal 300 mm from tip 32 might be employed because the first 300 mm of travel after insertion in a femoral artery are typically not of medical interest. In this case, adding 300 mm to the calculated displacement would give a total displacement of 767.3 mm.

### Exemplary Linear Displacement Sensor Types: Mechanical Sensor

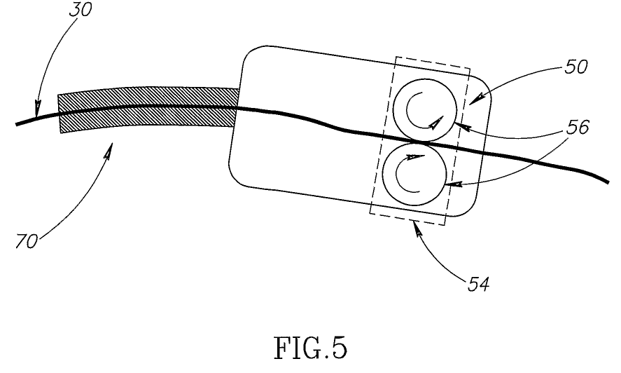

Referring now to FIG. 5, in an exemplary embodiment of the invention, linear displacement sensor 50 employs mechanical sensing means 54 such as, for example, one or more calibrated wheels 56 or gears that measure how much of catheter 70 and/or guidewire 30 has passed a given point. According to this embodiment of the invention, sensing is of the total number of forward turns of wheel(s) 56, and not of the object being measured (e.g. guidewire 30 and/or catheter 70) per se.

In an exemplary embodiment of the invention, wheels 56 have a 1 cm diameter and sensor 50 is sensitive to 3 degrees of rotation of wheels 56 to provide a measurement increment of approximately 0.56 mm. Smaller wheels and/or greater sensitivity to rotation can permit measurement of smaller incremental displacements.

In an exemplary embodiment of the invention, the degree of slippage between wheels 56 and the measured object is small enough that it does not introduce significant error into the measurement. Optionally, catheter 70 and/or guidewire 30 are marked with indentations or teeth which engage matching teeth/indentations on wheels 56 to increase friction and/or prevent slippage. Alternatively or additionally, one or more of wheels 56 are not part of a drive mechanism which impels catheter 70 and/or guidewire 30 forward, but are passively turned by catheter 70 and/or guidewire 30 as it passes across the wheels, optionally by means of indentations or teeth as described hereinabove. Whether wheels 56 drive catheter 70 and/or guidewire 30 or are driven by these objects, the number of revolution that wheels 56 turn can be detected and translated into a linear displacement of catheter 70 and/or guidewire 30 as long as the circumference of the wheels is known.

Mechanisms for detecting and recording a number of revolutions of a wheel are well known to those of ordinary skill in the art and can be incorporated into the context of the present invention (e.g. mechanisms available from W. M. Berg Inc., NY, N.Y., USA).

In an exemplary embodiment of the invention, wheels 56 are operated by a stepper-motor which moves guidewire 30 and/or catheter 70 in defined increments.

### Assembly

In an exemplary embodiment of the invention, a catheter 70 having a section of a known length 110 is attached to a section of guidewire 30 of a known length 100 and moves along the guidewire.

In an exemplary embodiment of the invention, a first sensor 50 is fixed at a defined location as detailed hereinabove and measures displacement of guidewire 30 relative to this defined location as detailed hereinabove. In an exemplary embodiment of the invention, a second sensor 50 is attached to a proximal portion of catheter 70. Optionally, attachment is at a known distance from the defined location of the first sensor. Alternatively or additionally, an initial distance between the second sensor and the first sensor may be determined. The initial distance may be determined, for example, by having each of the two sensors read a different portion of code 38 on guidewire 30. Alternatively or additionally, the initial distance between the two sensors 50 may be measured manually.

In an exemplary embodiment of the invention, assembly of catheter 70 and guidewire 30 includes alignment of their respective distal ends so that an initial distance 105 may be calculated using known catheter and guidewire lengths. Alternatively or additionally, a mark on guidewire 30 at a known distance from guidewire tip 32 may indicate a desired point of attachment for second sensor 50 of catheter 70.

Assembly of components may be at a manufacturing facility and/or at point of use. In an exemplary embodiment of the invention, the guidewire 30, catheter 70 and sensors 50 are supplied as an assembled pre-calibrated unit.

In an exemplary embodiment of the invention, radio-opaque markers on guidewire 30 and/or catheter 70 are employed for alignment and/or calibration. Optionally, the radioactive source is also radio-opaque. In an exemplary embodiment of the invention, radio opaque markers are deployed on guidewire 30 at known distances (e.g. every 50 mm) from tip 32 of guidewire 30. Once the position of tip 32 as a function of displacement is determined, the positions of all of these markers become known and can be displayed. Optionally, this display of radio-opaque markers along the path traveled by catheter tip 32 permits a distance between tip 72 of catheter 70 and each of the radio-opaque to be determined.

In an exemplary embodiment of the invention, a stent and/or PCTA balloon installed at or near tip 72 of catheter 70 serves as a radio-opaque marker and/or a radioactive marker.

Incorporation of a Radioactive Tracking Source into Guidewire:

A radioactive tracking source may be incorporated into a variety of existing tools, such as guidewire 30. Optionally, this facilitates tracking of the tool by tracking the source. However, use of multiple radioactive sources on a same guide wire or on multiple tools may cause mutual interference with tracking. In an exemplary embodiment of the invention, a single radioactive source is employed.

Incorporation may be at any desired location on guidewire 30, for example at or near the guidewire tip 32. The source of ionizing radiation may be integrally formed with, or attached to, a portion of guidewire 30. Attachment may be, for example by gluing or welding the source onto guidewire 30. Alternatively or additionally, attachment is achieved by supplying the source as an adhesive tag (e.g. a crack and peel sticker), radioactive paint or radioactive glue applicable to the guidewire. Optionally, the source of ionizing radiation is supplied as a solid, for example a length of wire including a radioactive isotope. A short piece of wire containing the desired isotope may be attached to the guidewire by inserting it into a groove on the guidewire and gluing it in place. This results in co-localization of the guidewire and the source of radiation. The source may be integrally formed with the guidewire by, for example, by co-extruding the solid source with the guidewire during the manufacture of the guide wire. Alternately, or additionally, the source of ionizing radiation may be supplied as a radioactive liquid which can be applied to a porous tip 32 of guidewire 30 and dried and/or solidified and/or absorbed. Regardless of the exact form in which the ionizing radiation source is supplied, or attached to the guidewire, it should not leave any significant radioactive residue in the body of the subject after removal from the body at the end of a medical procedure. In an exemplary embodiment of the invention, the amount of radioactive material employed for tracking can be low and the issue of significant radioactive residue is less important. In an exemplary embodiment of the invention, a large amount of radioactive material is employed, and the issue of significant radioactive residue is more important. In an exemplary embodiment of the invention, a standard guidewire 30 is incorporated into the context of the invention by attaching a radioactive source near tip 32.

In general, a guidewire for cardiovascular applications is a long and fine flexible spring used to introduce and position an intravascular catheter (Online Medical Dictionary; University of Newcastle upon Tyne <http://cacerweb.ncl.ac.uk>). Optionally, the guidewire has sufficient rigidity to allow it to be fed into a body through an opening, e.g. a port into an artery, and sufficient flexibility to allow it to navigate a path through the body (e.g. through the blood vessels). For orthopedic applications, a rigid guidewire may be employed.

In an exemplary embodiment of the invention, a guide sheath may be employed in addition to or instead of a guidewire. Guide sheaths may be used in the context of pulmonary and/or intracranial and/or orthopedic applications. In an exemplary embodiment of the invention, the guide sheath is curved, and one or more tracking sources are deployed at the curve. The guidewire or guide sheath may then serve as a track for a second object, such as a catheter as explained in greater detail hereinabove.

Catheter 70 optionally carries a PTCA balloon or stent near tip 72 and/or includes one or more dye injection ports near tip 72. Catheters 70 which deliver a stent may employ a radio-opaque stent which is optionally useful in calibration, for example calibration of a linear displacement of tip 72 of catheter 70. Alternatively or additionally guidewire 30 may have one or more radio-opaque portions for calibration, of a linear displacement of tip 32 of guidewire 30. Optionally, these radio-opaque portions are metallic and are deployed at known intervals along guidewire 30.

For those embodiments of the invention which employ a radioactive tracking source, 0.01 mCi to 0.5 mCi, optionally 0.1 mCi or less, optionally 0.05 mCi or less can permit accurate tracking. One isotope suited for use in this context is Iridium-192. A radioactive source of this type may be tracked, for example, by a system including three directional sensor modules which rely on angular detection acting in concert to determine a location of the radioactive source as detailed hereinabove.

Although the invention has been described in the context of a catheter and guidewire, optionally a cardiac catheter and guidewire, the scope of the invention is wide and encompasses paired tool combinations adapted to a wide variety of medical procedures including, but not limited to, those conducted in the heart, lungs, kidneys, brain, bones and gall bladder. Alternatively or additionally, the operative principles described hereinabove may be employed in other contexts including, but not limited to endoscopy and/or guided biopsy procedures. In an exemplary embodiment of the invention, an endoscope carrying a tracking source and camera is used to define one or more targets in terms of linear displacement as explained hereinabove. A medical tool, for example a biopsy sampler, is then propelled along the endoscope and stopped at linear displacements which were previously judged to be targets.

In the description and claims of the present application, each of the verbs “comprise”, “include” and “have” as well as any conjugates thereof, are used to indicate that the object or objects of the verb are not necessarily a complete listing of members, components, elements or parts of the subject or subjects of the verb.

Some exemplary systems and methods according to the present invention rely upon execution of various commands and analysis and translation of various data inputs. Any of these commands, analyses or translations may be accomplished by software, hardware or firmware according to various embodiments of the invention. In an exemplary embodiment of the invention, machine readable media contain instructions for registering linear displacement data of a second object on a position plot of a first object and/or performance of methods described herein. In an exemplary embodiment of the invention, a computer 60 executes instructions for the registration and/or the data acquisition.

The present invention has been described using detailed descriptions of embodiments thereof that are provided by way of example and are not intended to necessarily limit the scope of the invention. In particular, numerical values may be higher or lower than ranges of numbers set forth above and still be within the scope of the invention. The described embodiments comprise different features, not all of which are required in all embodiments of the invention. Some embodiments of the invention utilize only some of the features or possible combinations of the features. Alternatively or additionally, portions of the invention described/depicted as a single unit may reside is two or more separate physical entities which act in concert to perform the described/depicted function. Alternatively or additionally, portions of the invention described/depicted as two or more separate physical entities may be integrated into a single physical entity to perform the described/depicted function. Variations of embodiments of the present invention that are described and embodiments of the present invention comprising different combinations of features noted in the described embodiments can be combined in all possible combinations including, but not limited to use of features described in the context of one embodiment in the context of any other embodiment. The scope of the invention is limited only by the following claims.

All publications and/or patents and/or product descriptions cited in this document are fully incorporated herein by reference to the same extent as if each had been individually incorporated herein by reference.

1. A method of determining a position of a second object which travels along a first object, the method comprising:
(a) determining a position for the first object;
(b) determining a linear displacement of the second object relative to the first object said displacement being along said first object; and
(c) ascertaining a position of said second object based upon said relative linear displacement and said position of said first object.

2. A method according to claim 1, additionally comprising measuring a linear displacement of said first object.

3. A method according to claim 2, wherein said linear displacement of said first object and said second object linear displacement are each determined relative to a defined point.

4. A method according to claim 3, wherein the defined point employed for said first object and said second object are a single defined point.

5. A method according to claim 3, wherein the defined point employed for said first object and said second object are two separate defined points and the method additionally includes computing a linear distance between a first defined point employed in determining said first object linear displacement and a second defined point employed in determining said second object linear displacement and correcting said second object linear displacement value by said linear distance.

6. A method according to claim 2, wherein said determining said linear displacement and said position for the first object is repeated to determine a series of linear displacements with correlated positions, said series of correlated positions defining a path.

7. A method according to claim 1, wherein said determining a linear displacement value for said second object is relative to a defined point on said first object.

8. A method according to claim 1, wherein said second object is a catheter.

9. A method according to claim 1, wherein said first object is a guidewire.

10. A method according to claim 9, wherein said guidewire carries a tracking source at or near its distal tip.

11. A method according to claim 10, wherein said tracking source is a radioactive source.

12. A system for performing a medical procedure, the system comprising at least two axially extensible members at least partially insertable within a body, at least one of said axially extensible members marked with an array of machine readable markings configured to aid in determination of relative linear displacement along a common path of said at least two axially extensible members with respect to one another, and a sensor adapted to operate on a portion of one of said axially extensible members, said portion being adapted to be located outside of said body.

13. The system of claim 12, wherein said machine readable markings designate length increments.

14. A system according to claim 12, additionally comprising a tracking source positioned at a distal portion of one of said axially extensible members.

15. A system according to claim 14, wherein said tracking source is a radioactive source.

16. A system according to claim 12, wherein said machine readable markings include a binary code.

17. A system according to claim 12, wherein said axially extensible members include a catheter.

18. A system according to claim 12, wherein said axially extensible members include a catheter guidewire.

19. A system according to claim 18, wherein said catheter guidewire bears said array of machine readable markings.

20. A system for determining a position of a second object and a first object which travel along a common path, the system comprising:
(a) a first object comprising a tracking source, said first object subject to displacement along a path;
(b) a second object subject to displacement along said first object; and
(c) a displacement sensor designed and configured to determine a relative displacement of said second object and said first object along said path.

21. A system according to claim 20, additionally comprising circuitry to convert said relative displacement of said second object and said first object along said path to a position of said second object.

22. A system according to claim 20, wherein said tracking signal includes radioactive disintegrations.

23. A system according to claim 20, wherein said displacement sensor includes an optical sensing mechanism.

24. A system according to claim 23, wherein said optical sensing mechanism reads a binary code.

25. A system according to claim 20, wherein said displacement sensor includes a mechanical sensing mechanism.

26. A system according to claim 20, wherein said first object includes a guidewire.

27. A system according to claim 20, wherein said second object includes a catheter.

28. A method of determining a position of a second object which travels along a first object, the method comprising:
(a) determining a path traveled by at least one point on the first object;
(b) causing the second object to travel along said path while additionally determining a linear displacement along said first object of at least one point on said second object relative to at least one point of said first object; and
(c) determining a position of a selected portion of the second object by calculating a progress along the path of the at least one point on the first object.

29. A method according to claim 28, wherein said calculation relies upon said linear displacement.

30. A method according to claim 28, wherein the said progress along the path of the first object is determined by:
(a) employing a fixed and known length for at least a portion of each of the first and second objects; and
(b) measuring a relative displacement of said at least one point on said second object along said first object.

31. A method according to claim 30, wherein said measuring said relative displacement is conducted outside a body of a subject.

32. A method according to claim 28, wherein the said linear displacement is determined by:
(a) defining a first object reference point at a known distance from a distal extremity of said first object;
(b) defining a second object reference point at a known distance from a distal extremity of said second object; and
(c) measuring a distance between said first object reference point and said second object reference point as a means of computing a relative position of said distal extremity of said first object and said distal extremity of said second object along said path.

33. A method according to claim 32, wherein said first object reference point and said second object reference point are initially aligned in a same position.

34. A method according to claim 28, wherein said second object is a catheter.

35. A method according to claim 28, wherein said first object is a guidewire.

36. A method according to claim 35, wherein said guidewire carries a tracking source at or near its distal tip.

37. A method according to claim 36, wherein said tracking source is a radioactive source.

38. A guidewire comprising a source of radiation integrally formed with or attached to a distal portion thereof, wherein said radiation is 0.5 mCi or less.

39. A guidewire according to claim 38, wherein said radiation is in the range of 0.01 mCi to 0.5 mCi.

40. A guidewire according to claim 38, wherein said detectable amount is 0.1 mCi or less.

41. A guidewire according to claim 38, wherein said detectable amount is 0.05 mCi or less.

42. A guidewire according to claim 38, wherein said isotope is Iridium-192.

43. A guidewire according to claim 38, wherein said radiation is gamma radiation.
